(19) United States
(12) Patent Application Publication
(10) Pub. No.: US 2021/0260245 A1
(43) Pub. Date: Aug. 26, 2021

(54) ACELLULAR DERMAL MATRIX, IN PARTICULAR FOR A MAMMARY PROSTHESIS

(71) Applicant: MECCELLIS BIOTECH, LA ROCHELLE (FR)

(72) Inventors: Anthony PERES, DOMPIERRE SUR MER (FR); Guillaume HOFMANSKI, MIEUXCE (FR); Aurélie LAMMELIN, RICHEBOURG (FR)

(21) Appl. No.: 17/254,573

(22) PCT Filed: Jun. 21, 2019
(86) PCT No.: PCT/EP2019/066522

§ 371 Date: Dec. 21, 2020

(30) Foreign Application Priority Data
Jun. 22, 2018 (FR) 1855590

(51) Int. Cl. A61L 27/36; A61L 27/50

(52) U.S. Cl. CPC A61L 27/362; A61L 2430/04; A61L 27/50; A61L 27/3683

(57) ABSTRACT

The object of the invention is an acellular dermal matrix (1) comprising a solid and flexible sheet (10) which is provided with a plurality of through-openings (4) and is characterized in that the openings (4) have an elongated shape and, in particular, are forming slits (40). The openings are particularly suitable for opening in the form of a diamond.

The invention relates to an acellular dermal matrix, in particular for covering a mammary implant in breast surgery.

The invention will be described more particularly with respect to a mammary prosthesis for breast reconstruction, but without being limited thereto. It is applicable to any acellular dermal matrix which is intended for covering a prosthesis with a relief, in particular having at least a domed shape.

In breast reconstruction following a mastectomy, or within the context of another surgical operation for aesthetic breast reconstruction, at present an acellular dermal matrix is used more and more, which covers the implant/the prosthesis, and which is connected to the pectoralis major muscle, thus avoiding the need to previously stretch the skin and the muscles before placement of the implant. Furthermore, a matrix of this kind helps in supporting the tissues and in their regeneration. An acellular dermal matrix comprising a flexible sheet, produced for example from porcine tissue, said sheet comprising a plurality of round through-holes, is known from American patent application US2017/0224460. The holes allow fluid to flow, and can be used for fixing suturing means.

The invention aims to propose another perforated acellular dermal matrix.

According to the invention, the acellular dermal matrix comprises a solid and flexible sheet which is provided with a plurality of through-openings, according to the thickness of the sheet, and is characterized in that the openings are elongate in shape, in at least one direction, and in particular form slots at least in the rest state of the matrix.

The “rest state of the matrix” means the state in which it is not subjected to any stretching.

In the rest state of the matrix, or in the stretched state, the shape of the openings is oblong.

Thus, the elongate shape of the openings allows for stretching in all directions of the sheet, which allows the matrix to perfectly fit the receiving surface of said matrix in the use position. The cross section of the openings is thus variable, in order to adjust to different traction effects, without the risk of creases on the surface of the matrix, or undesired tensions. The configuration of the openings and intermediate solid regions of the matrix allows for optimal shaping of the matrix onto the implant (the receiving surface), whatever the size.

According to the invention, the openings, which change from slots to apertures in the stretched position of the matrix, remain oblong in shape.

According to one feature, the openings are capable of opening/extending/stretching (in the stretched state of the matrix), according to a geometry that is polygonal in cross section, in particular in the form of a lozenge. Other geometries are conceivable, such as a star shape, originating from a cut that is elongate in at least one direction.

According to another feature, the openings have a cross section that is variable in size, in the stretched state of the matrix. This size depends on the relief to which the matrix fits, when covering a prosthesis in particular.

Preferably, the openings (the slots) are all the same shape in the rest state of the matrix.

The openings in the form of slots (at least in the rest state of the matrix) have a length of between 5 and 15 mm, preferably on the order of 10 mm. A slot in the rest state of the matrix preferably has a width of a most 1 mm. This width corresponds to the width of the cutting knife.

The openings/slots originate from a cut of the sheet, using suitable cutting means, for example of the punch, knife, or cutter type.

The elongate shape of the openings/slots in the rest state of the matrix is such that the expansion ratio of the width of the openings is preferably at least 2 to 1 in the stretched state of the matrix, in particular between 2:1 and 5:1.

Advantageously, the openings are distributed in columns, in particular being separated by a distance of between 2 and 5 mm, preferably on the order of 3 mm for one column, and on the order of 2 mm for two adjacent columns.

The openings form a mesh.

According to another feature, the openings are arranged on the sheet in a localized region, preferably so as to be central with respect to one of the dimensions of the sheet (in particular so as to be central with respect to the width of the sheet when it is rectangular, or at least when it is elongate in shape), and preferably the sheet comprises solid regions within the localized region of the openings, in particular solid regions forming regularly spaced longitudinal strips, preferably having a width of between 5 and 15 mm, preferably without extending over the entire length of the localized region when said localized region is oblong.

Advantageously, the localized region provided with openings comprises longitudinal solid regions extending, in length, in the longitudinal direction of the sheet, and, in other regions, solid portions, in particular substantially triangular in shape, which are arranged so as to be symmetrically opposite the region of widest part of the localized region, in a direction transverse to the longitudinal solid regions. Preferably, solid portions, in particular substantially triangular in shape, may furthermore be arranged symmetrical opposite the region of the widest part of the localized region (and above and to each side of the longitudinal solid strips), in a direction transverse to the longitudinal solid strips.

The openings have a regular mutual spacing apart from in the localized regions, which are solid and the length and width dimensions of which are much larger than the spacing between the openings. These localized solid regions are preferably in the shape of longitudinal strips, mentioned above, or solid triangular portions. Thus, these solid regions within the localized region of the openings form ruptures of the regular pattern of the openings.

The sheet is elongate in shape, in a longitudinal direction, and has two opposite parts according to a median plane transverse to the longitudinal direction. The first part is completely solid, and the second part is solid and provided with openings in a localized region.

The two parts preferably have a contour shape that is symmetrical with respect to the transverse median plane.

The sheet is for example rectangular in shape, and is intended to be cut in the region of the edge thereof, so as to fit the curved lines of a prosthesis when the matrix surrounds the prosthesis during use thereof.

The two parts of the sheet each have for example a curved contour, in an ovoid shape or in a shell shape. The two parts, having a curved peripheral line, meet in the region of the median plane, in a narrowed cross section.

The localized region provided with openings is on just one of the two parts and is centered with respect to the edge of said part.

According to an advantageous feature, the openings are arranged on the sheet in a localized region forming an ovoid surface, the localized region provided with openings comprising solid strips which are spaced apart and extend in the longitudinal direction, proceeding from the narrowest end of the ovoid surface, preferably without extending as far as the opposite end, and preferably solid portions, in particular substantially triangular in shape, being arranged symmetrically opposite the region of the widest part (transverse to the length) of the ovoid shape.

The invention also relates to an assembly comprising an acellular dermal matrix cited above, according to the invention, and a prosthesis, in particular mammary, which is covered completely or in part by the matrix, in particular the openings being arranged in a localized region which corresponds to a relief surface of the prosthesis.

The solid part of the flexible sheet (i.e. outside of the perforated localized region) has dimensions suitable for covering all or some of the prosthesis. The perforated localized region has a geometry and dimensions that are adjusted to the shape of the prosthesis.

The present invention will now be described with reference to examples, which are given for illustrative purposes only and in no way limit the scope of the invention, and on the basis of the accompanying drawings, in which:

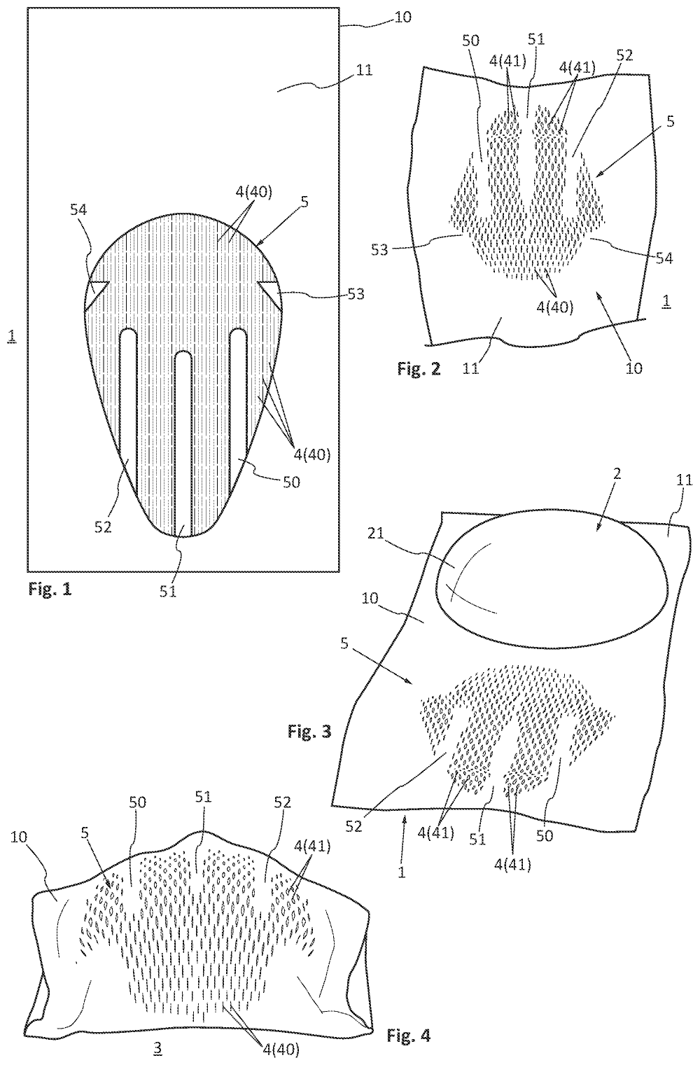

FIG. 1 is a schematic view from above of an embodiment of the acellular dermal matrix according to the invention, in the rest state;

FIG. 2 shows the matrix of FIG. 1 in a stretched state;

FIGS. 3 to 5 are perspective views of various successive steps of associating the matrix with a mammary prosthesis;

FIG. 6 shows the matrix assembly surrounding the prosthesis, the assembly being ready to be fixed;

FIG. 7 schematically shows the assembly of FIG. 6 in the fixed position;

FIGS. 8 and 9 are schematic cross sections of a matrix surrounding some or all, respectively, of a prosthesis;

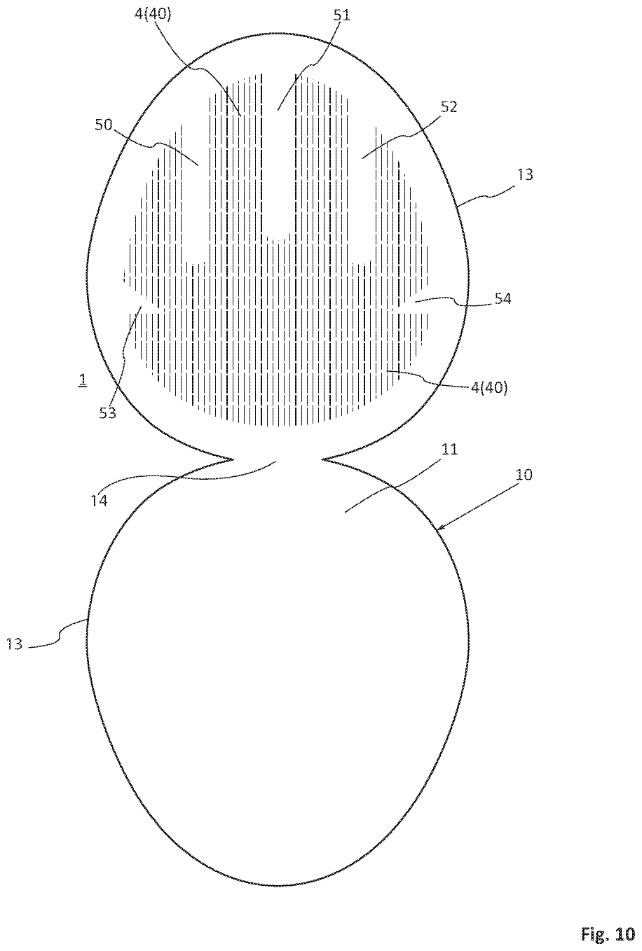

FIG. 10 is a variant of FIG. 1, in terms of the shape of the edge contour of the sheet.

The acellular dermal matrix 1 according to the invention, shown in the drawings, is intended in particular to surround a prosthesis 2, such as a mammary prosthesis, in order to form an assembly 3 which is intended to be connected to the pectoralis major muscle during mammary reconstruction on a patient.

The matrix 1 has dimensions suitable for the final use, both to cover the prosthesis 2 and to correspond to the receiving dimensions of the surface to be replaced by the prosthesis-matrix assembly.

The matrix 1 is of a size that is suitable for surrounding just a part of the prosthesis (FIG. 8) or the entirety of the prosthesis (FIG. 9).

The matrix 1 is generally of a size that is much larger than the prosthesis and, once the prosthesis is surrounded, the edge of said matrix is cut to the dimensions and geometry desired for the assembly 3.

In the embodiment shown, the matrix 1 is of a generally flat shape, which is rectangular (FIGS. 1 to 3).

The matrix 1 is made, in a known manner, of a flexible acellular dermal material, for example from porcine dermis.

According to the invention, the matrix 1 comprises a solid flexible sheet 10 which comprises a plurality of openings 4 which are spaced apart in a localized region 5, the openings 4 being elongate in shape.

Outside of the perforated localized region 5, the sheet 10 is solid and in particular comprises a solid receiving region 11 which is intended for placement of the prosthesis 2. The solid region 11 may be of different dimensions, depending on the prosthesis to be received and to be covered entirely or not.

With respect to FIGS. 2, 8 and 9, the prosthesis 2 comprises a face referred to as lower 20 which is intended to rest on/be associated with the receiving region 11, and an opposite face referred to as upper 21 which is intended to be covered by the perforated localized region 5.

According to the invention, the openings 4 advantageously form longilineal slots 40 in the rest position of the matrix, when said matrix is not subjected to any stretching (FIG. 1). The slots originate from the localized cut of the thickness of the sheet, made by any suitable means.

In the stretched position of the matrix (FIGS. 2 to 7), the slots 40 are capable of opening in order to form wider apertures 41.

Thus, the geometry and dimensions of the openings 4 can change. The slots 40 are capable of opening by means of traction exerted on the matrix.

Depending on the traction exerted on the sheet, which depends on the relief/the shape which the perforated region 5 is to fit, the openings 4 may remain, in some cases, in the shape of slots 40, and, for others, open in order to form apertures 41 which are much wider than the slots, the opening cross section being variable depending on the site, and thus on the relief to which it fits.

Consequently, the openings 4 can transition from a geometry in the form of a slot 40, i.e. narrow and extending in length, to an expanded/stretched geometry in the form of polygonal apertures 41, in particular in the form of apertures having a (substantially) lozenge-shaped cross section.

In order to achieve the polygonal shape of the apertures, the contour of the cut of the slots is adjusted. Of course, the cut is made in a mainly longitudinal direction, but also in angular directions at the ends of the slot, in particular originating from the adjusted tapering of the cutting means.

The slots 40 are preferably all identical. They have, for example, a length on the order of 10 mm. The width of the slot corresponds to the thickness of the blade of the cutting means.

Due to the resilient function of the flexible sheet 10, the slots 40 are capable of enlarging in order to form the apertures 41 having larger dimensions, essentially according to the transverse dimension or angularly with respect to the length of the slot. For example, the enlargement ratio of the apertures 4 according to the width (dimension transverse to the length of the slot) is between 2:1 and 5:1.

The openings 4 of the matrix thus have a variable size/cross section.

The openings 4 form a mesh in the flexible sheet 10, in the manner of a net in the use position of the matrix 1 on the prosthesis 2.

In the use position of the matrix 1, i.e. by covering the prosthesis 2, and consequently stretching of the matrix, the openings 4 have variable dimensions/sizes, depending on their site in the region 5, and thus depending on their location on the prosthesis. Indeed, the size of the openings depends on the geometry/the relief of the prosthesis.

In the embodiment shown, the prosthesis is a mammary prosthesis which is domed in shape. The stretching of the openings 4 on the most convex part is much greater than on the edge, as shown in FIGS. 4 to 7.

Preferably, the apertures 4 are arranged in spaced columns. The slots 40 are aligned in columns, having the longitudinal axis thereof in the direction of the column.

Preferably, the slots 40 are distributed in an equidistant manner. Two adjacent columns are separated (from a solid portion) for example on the order of 2 mm. Two consecutive slots of the same column are separated (from a solid portion) for example by 3 mm, According to the invention, the localized region 5 of the openings 4 has a geometry and dimensions that are adjusted to the final destination of the matrix 1 (for example a breast), and/or to the shape of the prosthesis.

In this case, for a mammary prosthesis the localized region 5 is generally ovoid or pear-shaped.

The length of the openings 4 (slots/apertures) extends in parallel with or substantially in parallel with the largest extension of the localized region 5, in this case with the major axis of the ovoid localized region 5.

Moreover, the perforated localized region 5 preferably solid portions are wider than the solid spaces for separating the openings 4. As shown in the drawings, the flexible sheet 10 comprises for example at least three solid portions 50, 51 and 52 which form longitudinal strips extending from the narrowest edge of the localized region 5, without extending as far as the opposite end, as well as two additional opposing portions 53 and 54 which return towards the interior of the localized region, in a direction transverse to the major axis and in the region of the largest width of the ovoid region. The additional opposing portions 53 and 54 are for example triangular in shape.

Merely by way of example, and in no way in a limiting manner, the dimensions associated with the matrix are as follows:
rectangular flexible sheet 10 that is 400 mm in length (to surround the entirety of the mammary prosthesis) or 200 mm (partial surrounding of the rear of the mammary prosthesis), by 180 mm width,
localized region 5 of openings 4, ovoid in shape and having a major axis on the order of 190 mm, and a minor axis on the order of 115 mm.

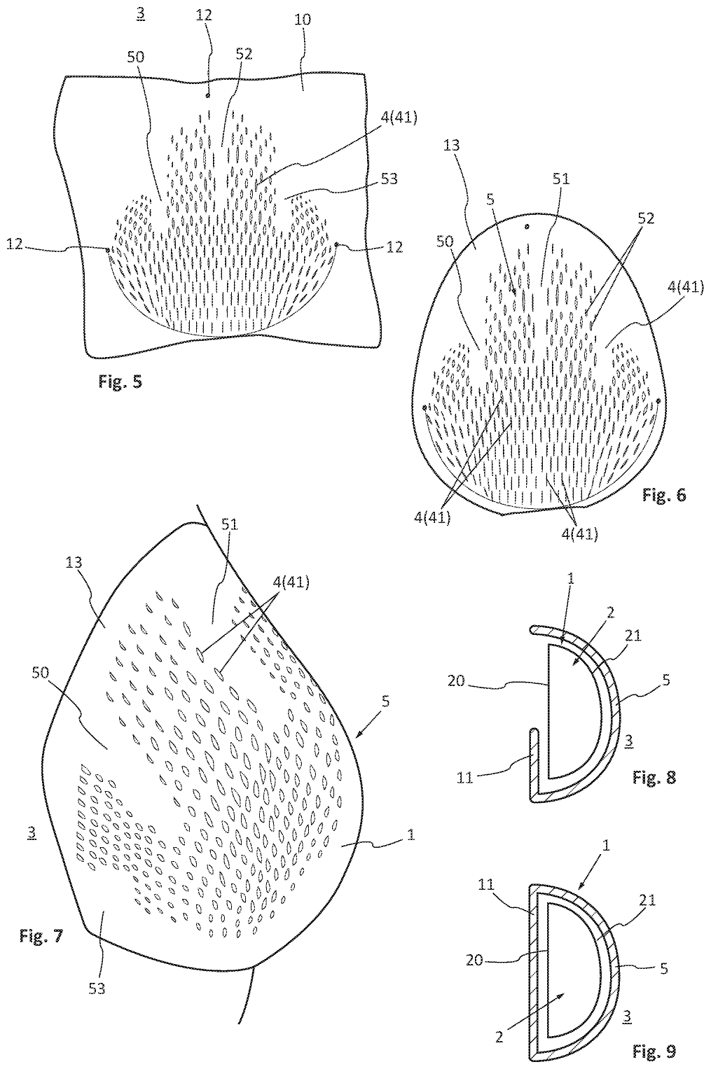

Forming the assembly 3 ready for use (matrix surrounding the prosthesis) is quick to implement. The steps are as follows:
the matrix 1 of FIG. 1 is plunged, flat, into a saline solution for approximately five minutes;
the sheet 10 of the matrix is then placed flat, as shown in FIG. 2, and the prosthesis 2 is placed down, by the lower face 20 thereof, on the solid region 11 of the sheet 10;
the sheet 10 is slightly stretched by the end sides peripheral to the perforated region 5, in order to be very tight;
the perforated region 5 is then returned to the upper face 21 of the prosthesis 2 in order to cover it entirely, as shown in FIG. 4;
a sewing step is performed at the edge 12 of the sheet 10 in order to form a closed pocked which keeps the prosthesis 2 in place, the assembly 3 is formed (FIG. 5);
preferably, the edge 12 of the assembly 3 is cut according to a contour 13 having a desired geometry (FIG. 6) with respect to its final destination.

The assembly 3 (acellular dermal matrix 1 and prosthesis 2) is ready to be positioned and fixed to the pectoralis major muscle, being capable of undergoing additional extension, as shown schematically in FIG. 7.

In the embodiment shown, the matrix 1 is of a generally flat shape, which is rectangular (FIG. 1 to 3).

In another embodiment of the matrix 1 (FIG. 10), the matrix 1 is of a generally flat shape which is elongate and in the form of two ovoid or shell-shaped parts. The edge perimeter of the sheet 10 has a contour 13 having a rounded line (and not straight, as in the example of the rectangular sheet) to be adapted directly to the shape of the prosthesis having a curved contour. The part provided with the localized region 5 of openings has a symmetrical contour 13 shape of the part forming the solid receiving zone 11, and according to a median plane transverse to the longitudinal direction of the sheet. Preferably, the two parts originating from the monolithic sheet 10 are formed in the region of a narrowed zone 14 of the median plane of symmetry. Thus, the matrix 1 of the invention, and therefore the assembly 3, allow for being stretched in a balanced manner over the entire contour 13 thereof, without creasing, by virtue of the surface adaptation of the openings 4 depending on the relief to which it is fitted. The matrix 1 perfectly fits the covered prosthesis 2, and does not cause any imbalance of stretching tension in the fixed position of the assembly 3.

1. Acellular dermal matrix (1) comprising a solid and flexible sheet (10) which is provided with a plurality of through-openings (4) and is characterized in that the openings (4) are elongate in shape, in at least one direction, in particular forming slots (40).

2. The acellular dermal matrix according to claim 1, characterized in that, in the rest state of the matrix, or in the stretched state, the shape of the openings (4) is oblong.

3. The acellular dermal matrix according to claim 1, characterized in that the openings (4) are capable of opening in the shape of a lozenge.

4. The acellular dermal matrix according to claim 1, characterized in that the openings (4) have a cross section that is variable in size, in the stretched state of the matrix.

5. The acellular dermal matrix according to claim 1, characterized in that the openings in the form of slots have a length of between 5 and 15 mm, preferably on the order of 10 mm.

6. The acellular dermal matrix according to claim 1, characterized in that the elongate shape of the openings (4) in the rest state of the matrix is such that the expansion ratio of the width of the openings is preferably at least 2 to 1, in particular between 2:1 and 5:1.

7. The acellular dermal matrix according to claim 1, characterized in that the openings (4) are distributed in columns, in particular being separated by a distance of between 2 and 5 mm, preferably on the order of 3 mm for one column, and on the order of 2 mm for two adjacent columns.

8. The acellular dermal matrix according to claim 1, characterized in that the openings (4) are arranged on the sheet (10) in a localized region (5), preferably so as to be central with respect to one of the dimensions of the sheet.

9. The acellular dermal matrix according to claim 1, characterized in that the sheet comprises solid regions (50 to 54) within the localized region of the openings, in particular the solid regions forming regularly spaced longitudinal strips (50, 51, 52), preferably having a width of between 5 and 15 mm, preferably without extending over the entire length of the localized region when said localized region is oblong.

10. The acellular dermal matrix according to either claim 8, characterized in that the localized region (5) provided with openings comprises longitudinal solid regions (50, 51, 52) extending, in length, in the longitudinal direction of the sheet, and, in other regions, solid portions (53, 54), in particular substantially triangular in shape, which are arranged so as to be symmetrically opposite the region of widest part of the localized region, in a direction transverse to the longitudinal solid regions.

11. The acellular dermal matrix according to claim 1, characterized in that the openings (4) are arranged on the sheet in a localized region (5) forming an ovoid surface, said localized region provided with openings in particular comprising solid strips (50, 51, 52) which are spaced apart and extend in the longitudinal direction, proceeding from the narrowest end of the ovoid surface, preferably without extending as far as the opposite end, and preferably solid portions 54), in particular substantially triangular in shape, being arranged symmetrically opposite the region of the widest part of the ovoid shape.

12. The acellular dermal matrix according to claim 1, characterized in that the sheet is elongate in shape, in a longitudinal direction, and has two opposite parts, according to a median plane transverse to the longitudinal direction, the first part (11) is completely solid, and the second part is solid and provided with openings in a localized region (5).

13. An assembly (3) comprising an acellular dermal matrix (1) according to claim 1, and a prosthesis (2), in particular mammary, which is covered completely or in part by the matrix, in particular the openings (4) being arranged in a localized region (5) which corresponds to a relief surface of the prosthesis.
